(19) United States
(12) Patent Application Publication
(10) Pub. No.: US 2006/0039948 A1
(43) Pub. Date: Feb. 23, 2006

(54) Implant, therapeutic agent and micelle

(76) Inventors: Gunther Schmid, Velbert (DE); Sandra Kipke, Stuttgart (DE)

Correspondence Address: WOODARD, EMHARDT, MORIARTY, MCNETT & HENRY LLP, 111 MONUMENT CIRCLE, SUITE 3700, INDIANAPOLIS, IN, 46204-5137, US

(21) Appl. No.: 10/535,517

(22) PCT Filed: Sep. 23, 2003
(86) PCT No.: PCT/EP03/10566

§ 371 Date: Aug. 17, 2005

(30) Foreign Application Priority Data
Nov. 14, 2002 (DE) 102 53 326.1
Nov. 22, 2002 (DE) 102 54 801.3

(51) Int. Cl. A61F 2/00

(52) U.S. Cl. 424/423

(57) ABSTRACT

The invention relates to an implant, a therapeutic agent, the use of micelles, which are formed from both surfactants as well as active substance molecules, and to a micelle. In order to achieve a defined delivery behavior through pores of a diffusion element, the active substance molecules are covered with surfactants.

The present invention concerns an implant according to the preamble of claim 1, a therapeutic agent according to the preamble of claim 19, a use of micelles formed from surfactants and active substance molecules, and a micelle.

The term “implant” is used here to mean, first in the strict sense, an element to be at least temporarily employed in the body of an animal or human being, which can have, for example, exclusively therapeutic functions, but also supporting and/or articulation functions. In the broader sense, however, it also means elements or such that can be brought into contact with the body from the outside, especially on a temporary basis.

The term “therapeutic agent” or “active substance” is used here to mean, in particular, drugs or pharmaceuticals, on the one hand, and curative agents and other substances administered to the human or animal body, on the other hand. In particular, all therapeutic agents or receptor agonists, receptor antagonists, enzyme inhibitors, neurotransmitters, cytostatics, antibiotics, hormones, vitamins, metabolic substrates, antimetabolites, diuretics, and the like, which are mentioned in EP 0 875 218 A2 and are called “medication” there, count as therapeutic agents.

The term “micelle” is used here to mean in the strict sense those aggregates that are formed from surfactant molecules in aqueous solutions above a particular temperature and a characteristic concentration—the so-called critical micelle forming concentration. In the broader sense it also means aggregates of dissolved molecules formed by association. In particular, it involves thermodynamically stable association colloids of surface-active substances wherein the hydrophobic residues of the monomers lie on the inside of the aggregates and are held together by hydrophobic interaction; the hydrophilic groups are turned toward the water and provide for the solubility of the colloid by salvation. In particular, micelles have characteristic aggregation numbers with usually just a slight range of distribution.

DE 199 48 783 A1, which forms the starting point for the present invention, discloses an implant with a receiving space for a therapeutic agent, which can dissipate out from the receiving space through an outlet element. For precise dispensing, the outlet element is proposed to be an open-pore diffusion element, in particular, one made from anodically oxidized aluminum oxide, and the walls of the pores can be chemically modified in order to control the diffusion.

When a therapeutic agent is used that has very small molecules of active substance, with diameters much less than the diameter of the pore, one can achieve a nearly free flow of the active substance molecules through the pores of the outlet element. Then, a precise control of the release of the therapeutic agent or the active substance molecules of the therapeutic agent is no longer possible.

The fundamental problem of the present invention is to specify an implant, a therapeutic agent, a use of micelles formed from surfactants and active substance molecules, and a micelle, which enable a very precise and preferably pressure-independent dispensing of, in particular, very small active substance molecules of a therapeutic agent through pores which are substantially larger than the active substance molecules, so that, in particular, a very precise dispensing is possible for the smallest of quantities.

The above problem is solved by an implant per claim 1, a therapeutic agent per claim 19, a usage per claim 25, or a micelle per claim 28. Advantageous modifications are the subject of the subsidiary claims.

A basic notion of the present invention is to provide active substance molecules of the therapeutic agent with an envelope, especially a molecular one, preferably formed from surfactants, especially by forming micelles, in order to enlarge the size, especially the diameter, and thereby enable a better dispensed release through pores.

Depending on the desired release behavior, the envelopes or micelles have an at least essentially uniform size and/or shape or, alternatively, a size and/or shape that varies as needed.

According to an especially preferred embodiment, the envelopes or micelles are formed at least essentially spherical.

The diameter of the envelopes or micelles without the hydration shell is preferably around 1% to 30%, in particular 2% to 20%, and very preferably only up to 10%, and with the hydration shell it is around 10% to 50%, especially 20% to 40%, of the mean pore diameter. Thus, the resulting release behavior is essentially characterized by diffusion and not by a pressure-dependent free flow through the pores.

The present invention shall be explained in further detail below, referring to the preferred embodiment examples of the drawing. These show:

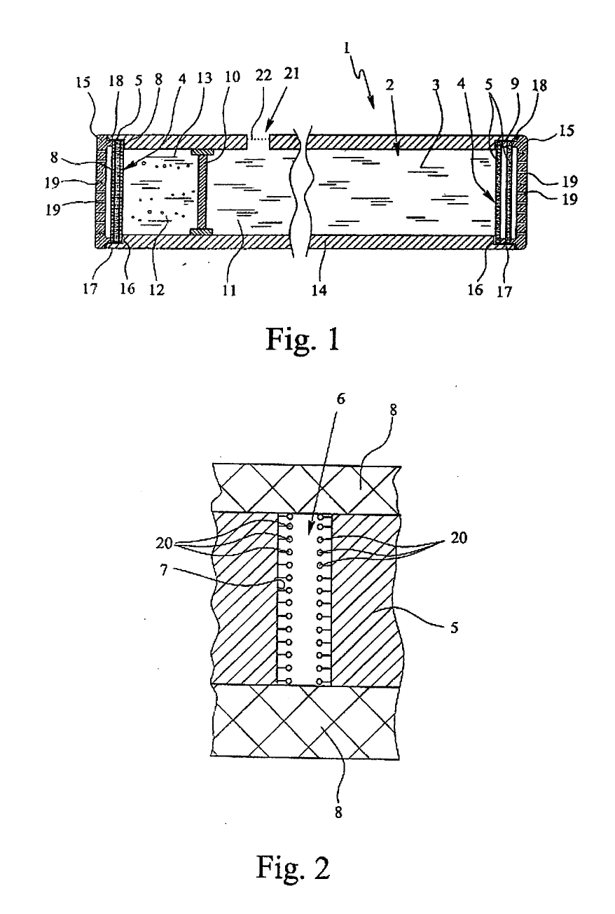

FIG. 1, a schematic cross section of a proposed implant;

FIG. 2, a schematic cross section of a pore of an outlet element of the implant per FIG. 1, supported on both sides;

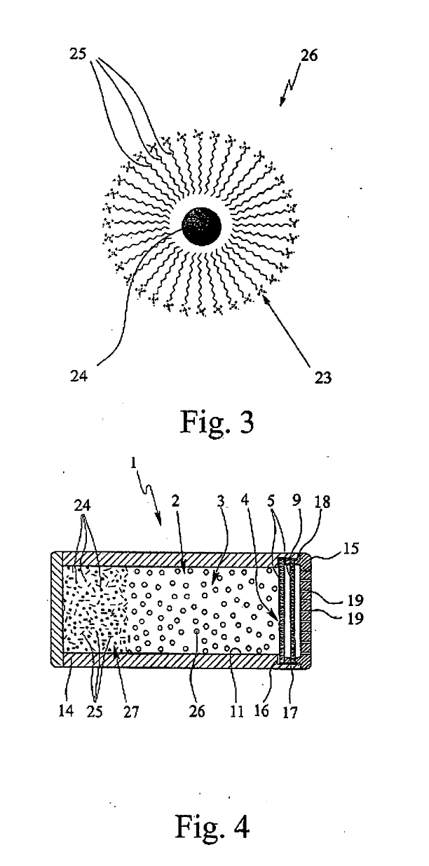

FIG. 3, a schematic view of an active substance molecule, provided with an envelope;

FIG. 4, a schematic cross section of a proposed implant according to another configuration variant; and

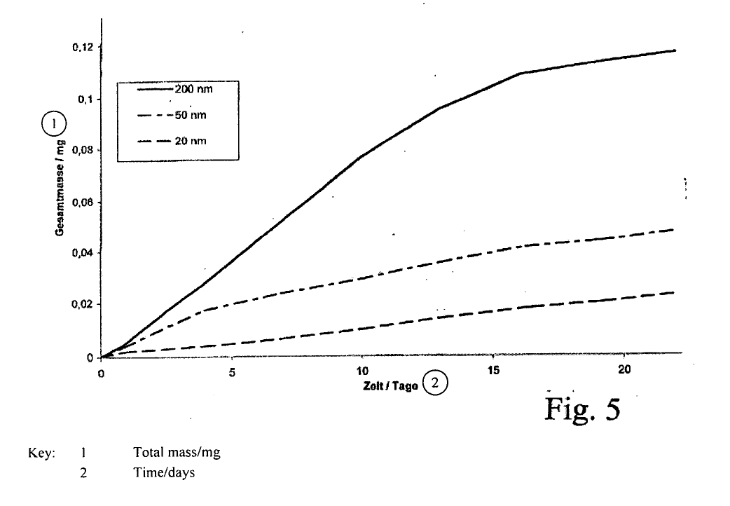

FIG. 5, a measurement diagram.

FIG. 1 shows an implant 1 in schematic view. The implant 1 has a receiving space 2 to hold a therapeutic agent 3. With respect to the therapeutic agent 3, refer to the definition in the introduction.

The implant 1 has at least one outlet opening 4, coordinated with at least one outlet element 5, which shall be explained in further detail by means of FIG. 2.

The outlet element 5 is permeable to the therapeutic agent 3 or at least one active substance of the therapeutic agent 3. For this, the outlet element 5 is preferably configured as an open pore. The outlet element 5 has a plurality of pores 6, through which the therapeutic agent 3 or at least an active substance of the therapeutic agent 3 can emerge to the outside 2, in particular, it can only diffuse through it.

Preferably, the surface density of the pores 6 is around 10⁸ to 10¹¹/cm². The mean pore diameter is preferably a maximum of 500 nm, in particular, 250 nm to 20 nm.

The outlet element 5 has a slight thickness of, in particular, less than 100 μm, especially around 50 to 70 μm, preferably at least 5 μm. Accordingly, there is a relatively low diffusion or flow resistance for the therapeutic agent 3 or at least for one active substance of the therapeutic agent 3.

The outlet element 5 preferably consists at least primarily of aluminum oxide, which is in particular formed or deposited electrolytically. However, the material for the outlet element 5 is not restricted to aluminum oxide, but instead all so-called valve metal oxides and magnesium oxide are generally usable. Besides these oxides, ceramic materials or other materials which have or enable a corresponding or different kind of pore formation—particularly by laser beam—are also generally usable.

The outlet element 5 is preferably supported on at least one side by at least one holding element 8, preferably configured as a lattice. FIG. 2 shows one configuration variant, in which the outlet element 5 is supported by a holding element 8 on both sides, i.e., it is held between two holding elements 8.

In the sample representation of FIG. 1, the implant 1 has a second opening 4, which is preferably arranged at the other, here, the left, end or opposite the first opening 4. This second outlet opening 4 is preferably also coordinated with an outlet element 5 as described above, so that an exchanging of material between the receiving space 2 of the implant 1 and the exterior space surrounding the implant 1 is also only possible through the outlet element 5.

In the sample representation of FIG. 1, only a single outlet element 5 is coordinated with the second outlet opening 4, being supported by holding elements 8 on both sides, as represented in FIG. 2.

On the other side, at the first opening 4, the two outlet elements 5 are kept apart from each other by a preferably ring-shaped spacing holder 9, as a configuration variant to the foregoing one.

As can be seen in FIG. 1, the implant 1 has a wall element 10, here essentially piston shaped, which divides the receiving space 2 into a first spatial section 11 and a second spatial section 12, the first spatial section 11 being in communication with the first or one outlet opening 4 and the second spatial section 12 being in communication with the second or another outlet opening 4. The wall element 10 is installed in the receiving space 2 so that it can slide like a piston here. However, a membrane or bellows type configuration of the wall element 10 can also be considered, given appropriate flexibility, mobility, and/or slidability.

The therapeutic agent 3 is preferably placed in only the first spatial section 11. A different agent, designated here as a compensation agent 13, is preferably contained in the second spatial section 12.

In particular, the outlet openings 4 are configured in the region of the ends, especially over the entire cross section of a hollow cylindrical base body 14 forming the receiving space 2. Moreover, protective covers 15 are coordinated with the outlet openings 4, especially to protect the outlet elements 5 against external mechanical influences.

In particular, a ring-shaped shoulder 16 is formed in the region of each outlet opening 4, abutting against which is a segment 17 with enlarged inner diameter of the base body 14 to accommodate the at least one outlet element 5 and corresponding holder element 8, spacing holder 9, or the like. The coordinated protective cover 15 has a cylindrical projection 18, which can be inserted into the segment 17 in a press fit.

The protective cover 15 has continuous openings 19, which have a large diameter as compared to the pores 6, so that an at least essentially undisturbed flow is possible through the protective cover 15.

The therapeutic agent 3 or at least one active substance of the therapeutic agent 3 can diffuse through the at least one outlet element 5 here through the two outlet elements 5 of the first outlet opening 4, which communicates with the first spatial section 11, and emerge into the body (not shown) surrounding the implant 1 through the continuous openings 19. The two outlet elements 5 of the first outlet opening 4 have pores 6 for this, whose pore size and/or pore wall 7 is configured such that, at least essentially, only a diffusion of the therapeutic agent 3 or the desired active substance of the therapeutic agent 3 emerges through the outlet elements 5 from the first spatial section 11 of the receiving space 2.

In order to achieve the aforesaid, preferably selective diffusion, the size of the pores 6 is adapted accordingly and/or the pore wall 7 is chemically modified accordingly by means of interaction partners 20, as indicated in FIG. 2. The interaction partners 20 are preferably fixed at least in regions on the pore wall 7 and provide, for example, a hydrophobic or hydrophilic property for the pores 6 or act as functional groups, in order to allow preferably only a selective passage through the outlet elements 5, i.e., to achieve essentially the action of a semipermeable membrane. The functional groups can be, for example, amine, mercapto, carboxy, hydroxy groups and/or organically modified silanes.

In order to compensate for the diminishing volume of the therapeutic agent 3 as therapeutic agent 3 or at least one active substance of therapeutic agent 3 is progressively released, the outlet element 5 of the second outlet opening 4, which communicates with the second spatial section 12 of the receiving space 2, is configured so that at least one substance, such as water, can penetrate from the body, not shown, surrounding the implant 1 through the outlet element 5 into the second spatial section 12 and possibly mix with the optionally provided compensation agent 13, such as a sodium chloride solution. Depending on the development of the outlet element 5 of the second outlet opening 4, this process of penetration can also occur without the compensation agent 13. In any case, the wall element 10, here configured so that it can slide, prevents an unwanted dilution of the therapeutic agent 3 and is moved into the spatial sections 1I and 12 as the volume changes.

It follows from the above that a kind of double osmosis occurs in the embodiment example depicted: on the one hand, the therapeutic agent 3 or at least one active substance of the therapeutic agent 3 emerges from the receiving space 2 and, on the other hand, a suitable substance enters into the receiving space 2 through the second outlet opening 4 or the outlet element 5 coordinated with it into the receiving space 2.

It further follows from the above that at least essentially only a diffusion of a suitable substance from the body, not shown, surrounding the implant 1 into the second spatial section 12 takes place. Thus, in particular, the outlet element 5 at this inlet side (left side of FIG. 1) is developed differently from the at least one outlet element 5 on the outlet side (right side in FIG. 1)—especially in terms of pore size, pore density and/or chemical modification of the pore walls 7.

If required, the implant 1 can have a septum 21, as indicated in FIG. 1. The septum 21 can serve for an initial filling and/or refilling of the therapeutic agent 3 or the compensation agent 13. If need be, two or more septa 21 can also be provided.

The septum 21 is an element already known in the state of the art, having a membrane 22 that can be pierced with an appropriately adapted cannula for filling or refilling the receiving space 2 and then automatically seals itself again.

In addition, reference is made to DE 199 48 783 Al, the entire contents of which is mentioned herewith as a supplemental disclosure of the present invention, especially as regards a preferred layout of the implant 1.

One major aspect of the present invention, now, consists in that the therapeutic agent 3 contains active substance molecules 24 provided with envelopes 23, especially molecular ones. These active substance molecules 24, in particular, form the primary relevant active substance of the therapeutic agent 3. The size and/or the shape, especially the diameter, of the envelope 23 is adapted to the size of the pores 6 in order to determine the release behavior. The envelope 23 consists at least essentially of surfactant(s) 25.

Preferably, the therapeutic agent 3 comprises an aqueous solution, wherein the active substance molecules 24 form micelles 26 with the envelopes 23. The micelles 26 are preferably configured to be at least essentially spherical.

The micelles 26 or envelopes 23 preferably have an at least essentially uniform size and/or shape.

The smallest, average, or largest diameter of the envelopes 23—without the hydration shell—amounts to at least essentially 2 to 200 nm, preferably 4 to 50 nm, and especially preferably 5 to 10 or 20 nm.

According to an especially preferred configuration variant, the size of the micelles 26 with hydration shell is at most ⅕, ¼, or ⅓ of the pore diameter.

FIG. 4 shows a configuration variant of the proposed implant 1, using the same reference numbers for the same or similar parts and components, and providing the same or at least similar benefits and characteristics, even though a repeated description is omitted for reasons of simplicity. In particular, the following only goes into particular differences.

The implant 1 has a solid reservoir 27 in the receiving space 2, which releases the therapeutic agent 3, or from which the therapeutic agent 3 can dissolve or be formed. In particular, the solid reservoir 27 consists of active substance molecules 24 and preferably surfactant(s) 25.

The solid reservoir 27 is in particular dissolved by the body's own fluids or other fluids in the receiving space 2 to form the therapeutic agent 3, so that the therapeutic agent 3 or active substances can then emerge or be dispensed through the outlet element 5 or the outlet elements 5 in desired manner, especially as is described above.

According to an especially preferred embodiment, the solid reservoir 27 is dissolved in such a way that the micelles 26 already described or a solution of micelles 26 is formed from the active substance molecules 24 and surfactants 25, preferably present as solids, in the receiving space 2.

The solid reservoir 27 has the benefit that an at least essentially constant concentration of active substances or micelles 26 or other substances can be maintained in the dissolved state in the receiving space 2 for much longer times, so that an essentially longer release time and/or a much more constant rate of release can be achieved than when the receiving space 2 has only a liquid fill.

Instead of or in addition to the surfactants 25, the solid reservoir 27 can also contain or comprise other suitable chemical substances that support or provide, in particular, a desired, preferably uniform dissolving of the active substance molecules 24 or other active substances.

The envelopes 23 or micelles 26, depending on the surrounding conditions and/or the active substance molecules 24 and surfactants 25 used, can be relatively dynamic, especially in terms of their shape, the number of associated molecules 24 and surfactants 25, and/or the exchange of the associated molecules 24 and surfactants 25.

The preferably at least essentially spherical envelopes 23 and micelles 26 can also become elongated and/or enter into other states of aggregation, for example, with lower aggregation numbers, as they pass through the pores 6. Nevertheless, the envelopes 23 and/or the formation of micelles has the effect that the diameter of the pores 6 constitutes an essential, in particular the determining, factor for the rate of diffusion of the therapeutic agent 3 through the pores 6 and, thus, for the rate of dispensing. The dispensing behavior is thus influenced, in particular, it is determined, in this way—at least for the most part.

The surfactant 25 can be, for example, SDS (sodium dodecyl sulfate). In particular, the surfactant 25 is chosen in dependence on or adapted to the active substance molecules 24.

The aggregation number of the micelles 26 is preferably at least 10, in particular at least 50, preferably at least 100, and very especially preferred around 150 or more. Accordingly, the molar ratio of the active substance molecules 24 to the surfactants 25, especially in the solid reservoir 27, is at least 1:10, especially at least 1:50, preferably at least 1:100, and very specially preferred around 1:150 or (possibly much) more.

According to a configuration variant not depicted, to improve the solubility of the solid reservoir 27 and/or to reduce the concentration gradients, a loose mixed body can be arranged in the receiving space 2, which has a much larger or smaller density than the therapeutic agent 3 in the receiving space 2 and accordingly moves around in the receiving space 2 during movements of the implant 1. If need be, several mixed bodies, such as spherical ones, for example, glass or ceramic balls, can also be provided in the receiving space 2 in order to improve the dissolving and/or mixing.

The diagram per FIG. 5 illustrates the results of an experiment. The total mass of active substance or active substance molecules 24 dispensed is plotted against the time of the experiment. Crystal violet was used as the active substance or for the active substance molecules 24 and SDS as the surfactant 25 in a solid reservoir 27. The molar ratio of crystal violet to SDS was around 1:150. An elongated receiving body was used, essentially corresponding to the implants 1 shown in FIGS. 1 and 4, but with only one outlet element 5.

In order to minimize concentration gradients in the receiving space 2 or make the release comparable, the sample and the receiving body were moved during the experiment, in particular, they were weighed.

The diagram per FIG. 5 shows that the dispensing rates depend considerably on the pore diameters (200 nm, 50 nm, 20 nm) of the different outlet elements 5 and vary accordingly. On the other hand, in control experiments with pure crystal violet, i.e., without the addition of surfactants 25, there was no dependency on pore diameter. Consequently, the envelopes 23 and the formation of micelles lead to the desired dependency of the dispensing rate on the pore diameter and, thus, to a controlling influence or determination thereof.

It should be noted that the presently invented enclosing or formation of micelles 26 is not confined merely to the use with implants 1, but rather can be used generally for any given diffision processes through pores 6, especially for the dispensing of active substances or active substance molecules 24.

1-30. (canceled)

31. Implant with a receiving space, a therapeutic agent contained therein, and an outlet element, wherein the outlet element has pores through which the therapeutic agent can leave the receiving space and be dispensed from the implant, wherein the therapeutic agent has or produces active substance molecules provided with a molecular envelope in order to influence the dispensing behavior.

32. Implant per claim 31, wherein the envelope consists at least essentially of surfactants.

33. Implant per claim 31, wherein the active substance molecules form micelles with the envelopes.

34. Implant per claim 31, wherein the size of the envelopes with the hydration shell amounts to at most ⅕ of the pore diameter.

35. Implant per claim 31, wherein the outlet element is configured as a diffusion element with open pores with a pore size or pore wall that at least essentially only allows for a diffusion of the ensheathed active substance molecules through the diffusion element, without enabling a free flow through the outlet element.

36. Implant per claim 31, wherein the outlet element has open pores with pore walls that are chemically modified at least in regions, in order to interact with the ensheathed active substance molecules with regard to passage through the outlet element.

37. Implant per claim 31, wherein the implant has a solid reservoir containing the active substance molecules and surfactant(s).

38. Implant per claim 37, wherein the active substance molecules can dissolve to form micelles.

39. Implant per claim 37, wherein the molar ratio of the active substance molecules to the surfactants in the solid reservoir is at least 1:50.

40. Therapeutic agent, wherein the therapeutic agent has or produces active substance molecules provided with molecular envelopes of surfactants.

41. Therapeutic agent per claim 40, wherein the therapeutic agent is an aqueous solution or a solid.

42. Therapeutic agent per claim 40, wherein the therapeutic agent is adapted for release through an implant of pores.

43. Therapeutic agent per claim 40, wherein the smallest, average, or largest diameter of the envelopes without the hydration shell is essentially 2 to 200 nm.

44. Therapeutic agent per claim 40, wherein the active substance molecules form micelles with the surfactants.

45. Method for modification of the diffusion behavior of active substance molecules, wherein micelles are formed from surfactants and the active substance molecules, and the active substance molecules are ensheathed by the surfactants.

46. Method per claim 45, wherein the smallest, average, or largest diameter of the micelles without the hydration shell is at least essentially 2 to 200 nm.

47. Method per claim 45, wherein the micelles are released through an implant or pores.

48. Micelle, comprising at least one active substance molecule, surrounded by an envelope formed from surfactant(s).

49. Micelle per claim 48, wherein the smallest, average, or largest diameter of the micelles without the hydration shell is at least essentially 2 to 200 nm.

50. Micelle per claim 48, wherein the micelles are essentially uniform in size or spherical in shape.
